(19) United States
(12) Patent Application Publication
(10) Pub. No.: US 2018/0021387 A1
(43) Pub. Date: Jan. 25, 2018

(54) PROBIOTIC COMPOSITIONS AND METHODS OF USE

(71) Applicant: WESTERN UNIVERSITY OF HEALTH SCIENCES, Pomona, CA (US)

(72) Inventor: Brian OAKLEY, Claremont, CA (US)

(21) Appl. No.: 15/547,974

(22) PCT Filed: Feb. 4, 2016
(86) PCT No.: PCT/US16/16587

§ 371 Date: Aug. 1, 2017

Related U.S. Application Data
(60) Provisional application No. 62/113,137, filed on Feb. 6, 2015.

(51) Int. Cl. A61K 35/57; A23K 20/10; C12N 1/20; A23K 50/75; A61K 9/00; A61K 35/741

(52) U.S. Cl. CPC A61K 35/57; A61K 9/0053; A61K 35/741; C12N 1/20; A23K 50/75; A23K 20/10; A61K 2035/115

(57) ABSTRACT

Compositions for oral administration to a young poultry bird, such as a chicken, that comprise cecal material from adult poultry birds and saline are disclosed. The compositions can be used to increase body weight gain in a poultry bird by administering them to a young bird by oral gavage. The compositions may also be used to increase feeding efficiency in a poultry bird. Further disclosed are compositions for application to poultry litter, for example, chicken litter, that are prepared using the cecal material from adult poultry bird. The compositions may be used to prevent or reduce colonization of the chicken gut by pathogenic bacteria. Also disclosed are method of selecting for bacterial strains with probiotic potential.

### FIELD OF THE INVENTION

This invention relates to probiotic compositions comprising cecal material from adult poultry and methods of their use.

### BACKGROUND OF THE INVENTION

Poultry feed costs on a live-weight basis have more than doubled in the last decade (according to National Agricultural Statistics Service), and they are expected to continue to increase in the future. Feed costs typically represent at least two-thirds of total production costs. Feed conversion ratios (FCR) and growth rates have been greatly improved by optimizations of feeding regimens, feed additives, and breeding, and important progress has also been made in understanding genetic factors underlying feed efficiency. However, room for additional improvements in these factors is seen as limited, and with feed costs likely to continue to increase from already high levels, FCR is a critical parameter for the success of the poultry industry. Water and land are the most important limitations to food supply and the production of greenhouse gasses is purported to be the biggest threats to global ecology.

Although poultry production is the most efficient form of terrestrial animal protein production, this is currently achieved by using in-feed antibiotics to manipulate the intestinal microbiota to optimize bird health and food safety. As antibiotic use in animal agriculture is increasingly negatively perceived by consumers and regulators, the time is ripe to utilize recent technological advances in the fields of microbial ecology and DNA sequencing to improve FCR by optimizing intestinal microbiota.

The importance of the gastrointestinal (GI) microbiota for nutrition and health of the animal host has been demonstrated by the efficacy of antibiotic growth promoters (AGPs) and their long history of use in agriculture. Although their exact mechanisms of action remain mostly unknown, the success of AGPs suggests that the GI microbiota modulates (and thus can be optimized to improve) nutrition and health. Poultry are naturally adapted to hosting a complex GI microbial community with hundreds of bacterial species and up to 100 billion cells per gram of gut contents.

Probiotics are very promising for their demonstrated ability to enhance performance, reduce reliance on antibiotics, and reduce colonization of the gut by pathogenic bacteria. It is possible to produce defined cultures of favorable probiotic organisms at a fraction of the cost of in-feed antibiotics and thus immediate benefits to the industry are possible for the relatively low cost of a bacterial inoculum. Therefore, currently there is a need to develop probiotic compositions that mimic the natural microbial community and methods of their use.

Long-range benefits are possible if this approach also limits pathogen colonization. This is conceptually similar to earlier versions of competitive exclusion (CE) products based on mixed cultures, with the important difference that any inoculum can now be completely defined using next-generation DNA sequencing.

The healthy chicken selects for a very specific and reproducible subset of the complex bacterial community to which it is exposed. Ecological theory suggests that each of these symbiotic bacteria have evolved a unique role in the ecology of the gastrointestinal tract.

Furthermore, current industry practices almost completely ignore the microbiology of the litter used in commercial poultry houses and its potential to influence the gut microbiota of broilers despite the fact that we now know that the litter harbors an abundant (>100 million per gram) and diverse (>200 bacterial genera) microbial community that is the primary source of the microbiota colonizing broiler chicks. Given the influences of the gut microbiota on bird performance, not managing the microbiology of litter leaves a major aspect of poultry husbandry entirely up to chance, which is not appropriate for a commercial production system. Managing the microbiology of the litter through sanitation treatments followed by application of defined probiotic formulations can provide great benefits to the industry.

Additionally, the regulatory process for probiotics provided as poultry feed additives or nutritional supplements can take years before a commercially viable product is approved. In sum, there is currently a need for probiotic compositions that can be applied to the litter to influence the gut microbiota of poultry. Such compositions and their use can have immediate and long-lasting benefits, such as improving food safety, increasing feed efficiency, and limiting pathogen colonization, in a more efficient manner and at a lower cost than using other methods (e.g., antibiotics).

### SUMMARY OF THE INVENTION

Disclosed herein, in certain embodiments, are compositions suitable for oral administration to a young poultry bird comprising cecal material from adult poultry birds and saline, wherein the ratio of the cecal material to saline ranges from 1:2 to 1:4 (v/v). In some embodiments, the cecal matter is from adult chickens, and the compositions are suitable for oral administration to a young chicken. In some embodiments, the ratio of the cecal matter from adult chickens and saline is 1:3.

Disclosed herein, in certain embodiments, are methods of increasing body weight gain in a poultry bird comprising administering to a young poultry bird by oral gavage a composition that includes cecal material from adult poultry birds and saline, wherein the ratio of the cecal material to saline ranges from 1:2 to 1:4 (v/v). In some embodiments, the disclosed methods of increasing body weight gain in a chicken comprise administering to a young chicken by oral gavage a composition that includes cecal material from adult chickens and saline, and the ratio of the cecal material to saline is 1:3. In some embodiments, the compositions of the invention are administered to a day-of-hatch chick.

Disclosed herein, in certain embodiments, are methods of increasing feeding efficiency in a poultry bird comprising administering to a young poultry bird by oral gavage a composition that includes cecal material from adult poultry birds and saline, wherein the ratio of the cecal material to saline ranges from 1:2 to 1:4 (v/v).). In some embodiments, the disclosed methods of increasing feeding efficiency in a chicken comprise administering to a young chicken by oral gavage a composition that includes cecal material from adult chickens and saline, and the ratio of the cecal material to saline is 1:3.

Disclosed herein, in certain embodiments, are compositions suitable for application to poultry litter, wherein the composition is prepared by (a) obtaining the cecal material from adult poultry birds; (b) administering the cecal material to young poultry birds by oral gavage; (c) obtaining the cecal material from the inoculated poultry birds and subjecting it to serial passaging through multiple generations of birds; (d) obtaining the serially-passaged cecal material; and (e) suspending the serially-passaged cecal material in saline.

Disclosed herein, in certain embodiments, are compositions suitable for application to the chicken litter, wherein the composition is prepared by (a) obtaining the cecal material from adult chickens; (b) administering the cecal material to young chickens by oral gavage; (c) obtaining the cecal material from the inoculated chickens and subjecting it to serial passaging through multiple generations of birds; (d) obtaining the serially-passaged cecal material; and (e) suspending the serially-passaged cecal material in saline.

Disclosed herein, in certain embodiments, are methods of preventing or reducing colonization of the chicken gut by pathogenic bacteria comprising the step of applying to the chicken litter a composition prepared by (a) obtaining the cecal material from adult chickens; (b) administering the cecal material to young chickens by oral gavage; (c) obtaining the cecal material from the inoculated chickens and subjecting it to serial passaging through multiple generations of birds; (d) obtaining the serially-passaged cecal material; and (e) suspending the serially-passaged cecal material in saline.

Disclosed herein, in certain embodiments, are methods of preventing or reducing colonization of poultry gut by pathogenic bacteria comprising the step of applying to the poultry litter a composition comprising: (a) cecal material from adult poultry birds and (b) saline, wherein the ratio of (a) to (b) ranges from 1:2 to 1:4 (v/v).

Disclosed herein, in certain embodiments, are litter compositions that include poultry litter and a composition comprising: (a) cecal material from adult poultry birds and (b) saline, wherein the ratio of (a) to (b) ranges from 1:2 to 1:4 (v/v).

Disclosed herein, in certain embodiments, are method of selecting for bacterial strains with probiotic potential comprising the steps of: a) mixing a suspension of cecal contents with chloroform to select for spore-forming bacteria and b) removing the chloroform from the mixture by centrifugation and washing with saline.

Disclosed herein, in certain embodiments, are compositions suitable for application to the chicken litter, wherein the compositions are prepared by (a) obtaining the cecal material from adult chickens; (b) administering the cecal material to young chickens by oral gavage; (c) obtaining the cecal material from the inoculated chickens and subjecting it to serial passaging through multiple generations of birds; (d) obtaining the serially-passaged cecal material; (e) suspending the serially-passaged cecal material in saline; (f) mixing the suspension of the cecal material with chloroform to select for spore-forming bacteria; (g) removing the chloroform from the mixture by centrifugation; and (h) washing with saline, wherein the composition comprises one or more of the following strains of Clostridia: c2-6-12; c1-8; m1-10-15; m2-9; mg20-4; m20-1; c20-1; and m20-5.

### BRIEF DESCRIPTION OF THE FIGURES

FIG. 1 depicts growth comparison of inoculated versus uninoculated chicks for males (M) and females (F) chicks. Chicks received a single inoculum (0.2 mL of a suspension of cecal contents from adult birds) at day of hatch and were reared in identical conditions essentially identical to commercial poultry rearing. Six weeks of age represents typical commercial harvest.

FIG. 2 depicts feed efficiency comparison of inoculated versus uninoculated chicks. Each point represent a single bird. Y axis values represent RFI calculated as the amount of feed consumed relative to the amount expected based on regression modeling. Lower values represent more efficient birds as less feed is consumed than expected. Difference of means is significant (p<0.001) by two-tailed t-test.

FIG. 3 shows comparison of DNA fingerprints of cecal microbiota at 6 weeks of age from inoculated versus uninoculated birds. Each point represents the cecal microbial community from a single bird. Plot represents a canonical correspondence analysis (CCA) in which the taxonomic profiles of each cecal community is the input. Proximity in graph space corresponds to similarity of the cecal microbiota.

FIG. 4 depicts taxonomic composition of the cecal microbiota at 6 weeks of age from inoculated versus uninoculated birds as classified to the genus level.

FIG. 5 shows 16S rRNA-based phylogenetic tree showing the nearest known relatives to eight of the isolates belonging to the Clostridiales family known to be important members of the poultry gut microflora isolated by novel method of exposing cecal contents to chloroform to select for spore-forming bacteria.

FIG. 6 shows growth inhibition of C. perfringens by the c2-6 and c1-8 novel isolates.

### DETAILED DESCRIPTION

The compositions described herein, in certain embodiments, are suitable for oral administration to young poultry birds and comprise (a) cecal material from adult poultry birds and (b) saline, wherein the ratio of (a) to (b) ranges from 1:2 to 1:4 (v/v).

As used herein, “poultry” are domesticated fowl, such as chickens, turkeys, ducks, or geese. The intestinal portion of poultry GI tracts includes serval parts, such as duodenum, upper jejunum, lower jejunum, ileum, and ceca. Generally, ceca are two blind pouches that are attached at the junction between the small intestine and the large intestine. As used herein, “cecal material” includes the content and scrapings of the ceca that can be combined, ground, and/or homogenized.

As used herein, “adult poultry” or “adult chickens” are birds that are 21 days from hatch or older, such as, for example, 28-day-old, 35-day-old, or 42-day-old. As used herein, a “young poultry bird” or a “young chicken” is a bird that is day-of-hatch to 7-day-old.

As used herein, “saline” is a solution of sodium chloride (NaCl) in water. In some embodiments, the concentration of sodium chloride in the solution is about 0.9% (w/v). Saline can be a buffered saline, such as phosphate-buffered saline (PBS) or tris-buffered saline. In some embodiments, the ratio of the cecal material to saline is 1:2, 1:2.25, 1:2.5, 1:2.75, 1:3, 1:3.25, 1:3.5, 1:3.75, or 1:4 (v/v).

In some embodiments, the cecal material of the compositions disclosed herein is from adult chickens, the ratio of the cecal material to saline is 1:3 (v/v), and the compositions are suitable for oral administration to a young chicken.

In some embodiments, the compositions can include other components, such as, for example, one or more of the following: a bacterial community significantly enriched in Bacteroides, a nutrient, an amino acid, a vitamin, a mineral, and a fatty acid. As used herein, a “nutrient” is a food or other substance that provides energy or building material for the survival and growth of a living organism. Examples of nutrients include, but are not limited to, carbohydrates, proteins, peptides, and fats. An “amino acid” can be any naturally-occurring or non-naturally-occurring molecule that contains both amine and carboxyl functional groups.

Disclosed herein, in certain embodiments, are methods of increasing body weight gain in a poultry bird comprising administering to a young poultry bird by oral gavage about 0.2 mL of a composition that comprises (a) cecal material from adult poultry birds and (b) saline, wherein the ratio of (a) to (b) ranges from 1:2 to 1:4 (v/v). Cecal material can be collected and combined from several adult poultry birds, for example from 2, 3, 4, 5, or more adult birds.

Unless otherwise specified, “oral gavage” is a technique used for delivering a liquid or a substance to the bird stomach through a tube, a needle, or a cannula.

Disclosed herein, In some embodiments, are methods of increasing body weight gain in a chicken comprising administering to a young chicken by oral gavage about 0.2 mL of a composition that comprises the cecal material from adult chickens and saline, wherein the ratio of the cecal material to saline is 1:3. In some embodiments, the young chicken is a day-of-hatch chick.

Disclosed herein, in some embodiments, are methods of increasing feeding efficiency in a poultry bird comprising administering to a young poultry bird by oral gavage about 0.2 mL of a composition that comprises (a) cecal material from adult poultry birds and (b) saline, wherein the ratio of (a) to (b) ranges from 1:2 to 1:4 (v/v).

Disclosed herein, In some embodiments, are methods of increasing feeding efficiency in a chicken comprising administering to a young chicken by oral gavage about 0.2 mL of a composition that comprises the cecal material from adult chickens and saline, wherein the ratio of the cecal material to saline is 1:3. In some embodiments, the young chicken is a day-of-hatch chick.

Disclosed herein, in some embodiments, are compositions suitable for application to poultry litter, wherein the composition is prepared by (a) obtaining the cecal material from adult poultry birds; (b) administering the cecal material to young poultry birds by oral gavage; (c) obtaining the cecal material from the inoculated poultry birds and subjecting it to serial passaging through multiple generations of birds; (d) obtaining the serially-passaged cecal material; and (e) suspending the serially-passaged cecal material in saline. As used herein, “poultry litter” or “chicken litter” is a mixture of any material used as bedding in poultry operations (e.g., wood shavings, sawdust, or straw that is spread on poultry house floors) and possibly poultry excreta, spilled feed, and/or feathers. As used herein, unless specified otherwise, “inoculated” poultry birds or chickens are those that have been orally administered any of the compositions disclosed herein.

Poultry cecal material can be obtained, for example, by removing the GI tract of birds, cutting out the ceca or cecum, and grinding, homogenizing, and/or combining the cecal contents and scrapings.

The selection by the chicken for a specific set of GI bacteria results from a complex immune-mediated dialogue between bacteria and host. This phenomenon is consistent and reproducible. The invention exploits this phenomenon by using serial passaging and thus circumvents the difficulties of defining specific culture media by using the poultry or chicken itself to select for a specific community that can be provided to young poultry birds or chicks to improve growth performance.

Disclosed herein, in some embodiments, are compositions suitable for application to chicken litter, wherein the composition is prepared by (a) obtaining the cecal material from adult chickens; (b) administering the cecal material to young chickens by oral gavage; (c) obtaining the cecal material from the inoculated chickens and subjecting it to serial passaging through multiple generations of birds; (d) obtaining the serially-passaged cecal material; and (e) suspending the serially-passaged cecal material in saline. In certain embodiments, the young chickens in step (b) are day-of-hatch chicks. The serial passaging of step (c) can be done through at least 3 sets of chicks.

Disclosed herein, in some embodiments, are methods of preventing or reducing colonization of the chicken gut by pathogenic bacteria comprising the step of applying the presently compositions to chicken litter. The compositions can be applied, for example, by mixing with or spraying them on the litter. For example, the compositions can be sprayed as an aerosol where the aerosol spray contains 10⁸ probiotic cells per cm². Bacteria that are pathogenic for poultry and/or humans include, but are not limited to, Clostridium difficile, Clostridium perfringens, Clostridium septicum, Salmonella, and Campylobacter.

Disclosed herein, in certain embodiments, are methods of preventing or reducing colonization of poultry gut by pathogenic bacteria comprising the step of applying to the poultry litter a composition comprising: (a) cecal material from adult poultry birds and (b) saline, wherein the ratio of (a) to (b) ranges from 1:2 to 1:4 (v/v). In some embodiments, the ratio of the cecal material to saline is 1:2, 1:2.25, 1:2.5, 1:2.75, 1:3, 1:3.25, 1:3.5, 1:3.75, or 1:4 (v/v). The compositions can be applied, for example, by mixing with or spraying them on the litter. In some embodiments, the compositions are applied by aerosol spraying directly on the litter, and the aerosol spray comprises about 10⁸ probiotic cells per cm².

Disclosed herein, in some embodiments, are litter compositions comprising poultry litter and a composition comprising: (a) cecal material from adult poultry birds and (b) saline, wherein the ratio of (a) to (b) ranges from 1:2 to 1:4 (v/v). In some embodiments, the ratio of the cecal material to saline is 1:2, 1:2.25, 1:2.5, 1:2.75, 1:3, 1:3.25, 1:3.5, 1:3.75, or 1:4 (v/v).

Disclosed herein, in some embodiments, are methods of selecting for bacterial strains with probiotic potential comprising the steps of: a) mixing a suspension of cecal contents with chloroform to select for spore-forming bacteria and b) removing the chloroform from the mixture by centrifugation and washing with saline. As used herein, “probiotics” or “bacterial strains with probiotic potential” are live microorganisms (e.g., bacteria) that confer a beneficial health effect on the host.

Disclosed herein, in some embodiments, are composition suitable for application to chicken litter, wherein the compositions are prepared by (a) obtaining the cecal material from adult chickens; (b) administering the cecal material to young chickens by oral gavage; (c) obtaining the cecal material from the inoculated chickens and subjecting it to serial passaging through multiple generations of birds; (d) obtaining the serially-passaged cecal material; (e) suspending the serially-passaged cecal material in saline; (f) mixing the suspension of the cecal material with chloroform to select for spore-forming bacteria; (g) removing the chloroform from the mixture by centrifugation; and (h) washing with saline, wherein the composition comprises one or more of the following strains of Clostridia: c2-6-12; c1-8; m1-10-15; m2-9; mg20-4; m20-1; c20-1; and m20-5.

### EXAMPLES

The following ingredients, processes, and procedures for practicing the compositions and methods disclosed herein correspond to that described above. The procedures below describe with particularity illustrative, non-limiting embodiments of the compositions and methods that involve cecal material from adult chickens and methods of making and uses thereof.

### Example 1

Growth Comparison of Chicks Inoculated with Microbiota of Adult Chickens Versus Uninoculated Chicks.

Several experiments were performed to validate the efficacy of an inoculum prepared from the cecal material of adult chickens. The cecal material was administered to day-of-hatch chicks.

To obtain the cecal material, GI tracts of adult birds were removed and ceca were cut out. Cecal parts were then homogenized as contents and scrapings, mixed 3:1 with phosphate buffered saline (PBS) and used to inoculate the day-of-hatch chicks by oral gavage at 0.2 ml per bird. Birds were weighed at weekly intervals and RFI values calculated on a per bird basis for the last two weeks of the experiment.

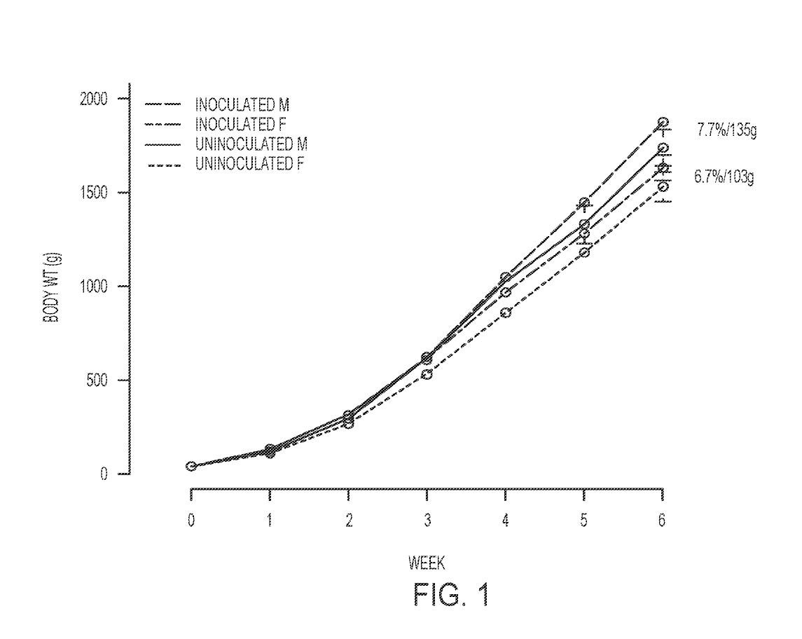

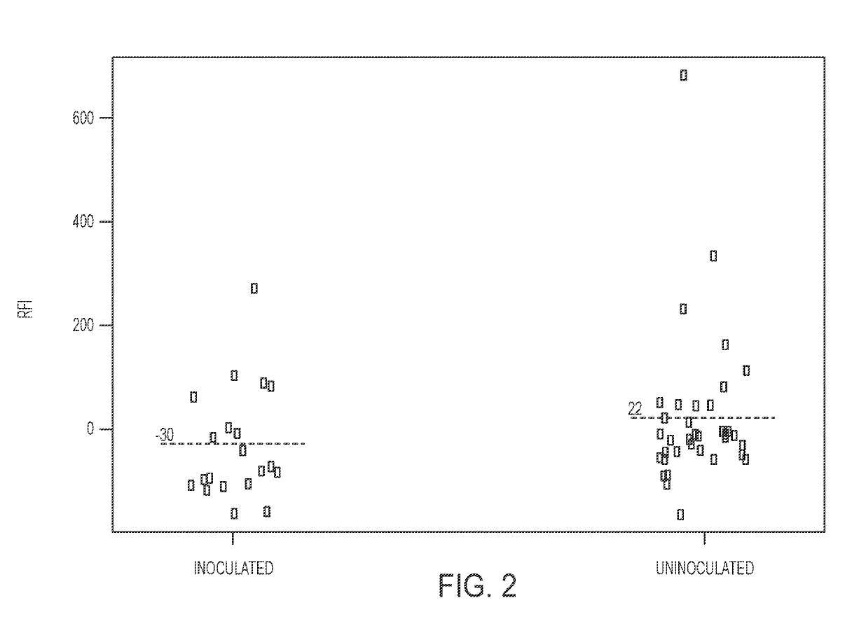

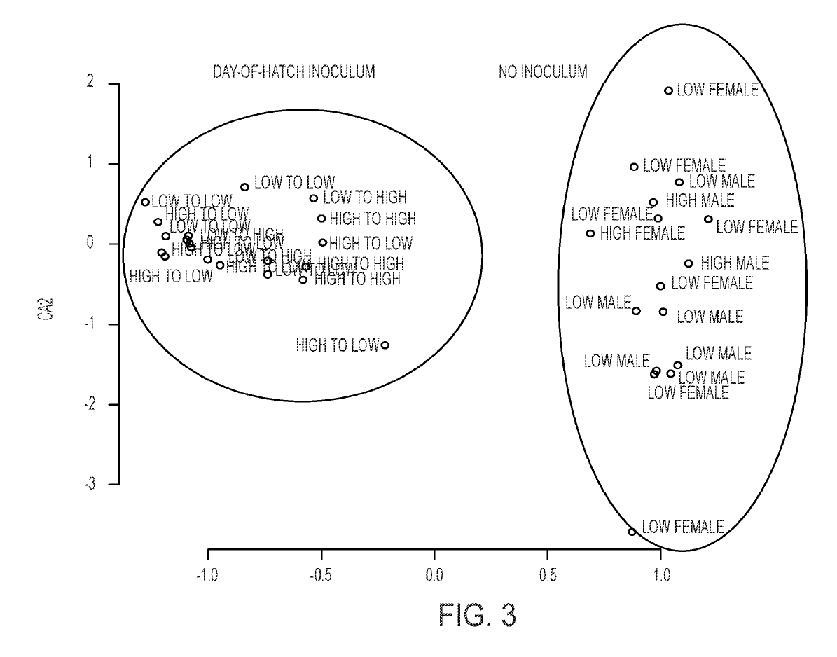

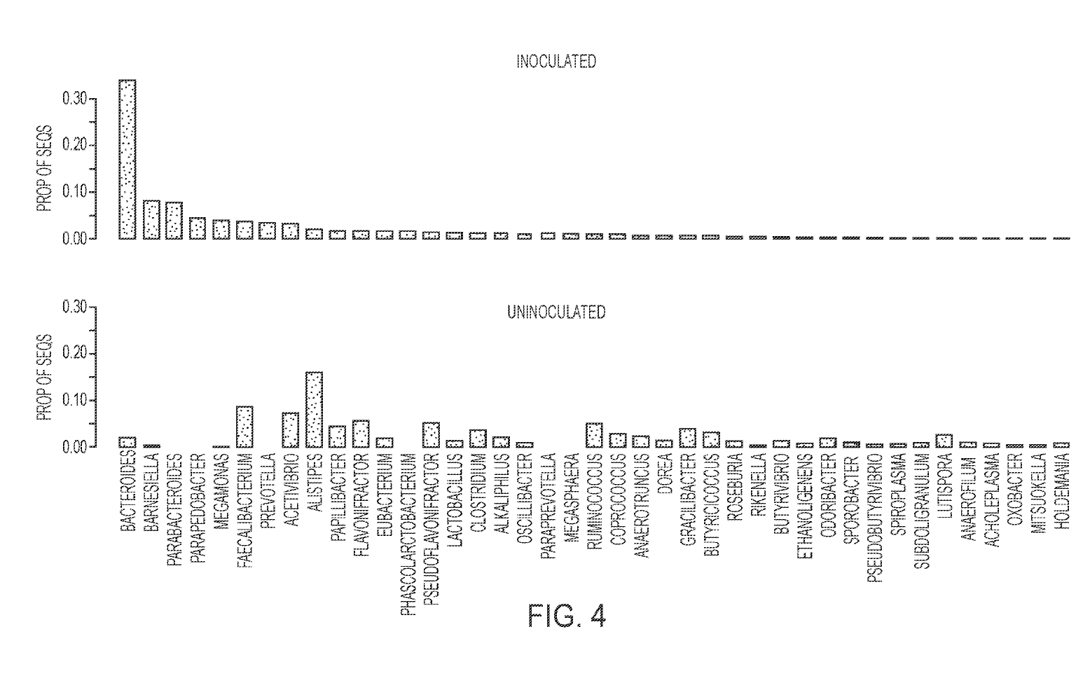

Approximately 80 newly-hatched chicks were divided into two groups—inoculated with the cecal material from adult chickens and uninoculated controls. The birds were then reared for 6 weeks in floor pens typical of industry conditions. Over the 6 week growth period, chicks that received an inoculum from adult birds showed significant improvements in body weight gain (FIG. 1) and feed efficiency (FIG. 2). They also developed significant differences in the taxonomic composition of the GI microbiota (FIG. 3 and FIG. 4), even 6 weeks after inoculation.

### Example 2

Identifying and Culturing Intestinal Probiotic Bacteria.

Samples were collected from the mid-gut and ceca of commercial chickens at 7, 21 and 41 days post-hatch. The samples were then treated with chloroform to eliminate vegetative bacterial cells and cultured under anaerobic conditions. Briefly, feces were suspended in phosphate-buffered saline (PBS) and mixed with chloroform while shaking at room temperature for 60 min. The chloroform was separated by centrifugation at 2,500 rpm for 20 minutes and the treated samples were drawn off with a pipette for culture. Samples were cultured on three different media and transferred until axenic colonies were obtained. Genomic DNA was sequenced on an Illumina MiSeq instrument with PE250 reads and data processed using standard genomics tools.

Approximately 40 isolates have been obtained and characterized in several ways. First, these isolates were tested to see if they could inhibit the growth of a variety of human and animal pathogens. Eight isolates from cecal samples and five from mid-gut samples inhibited the growth of at least one of C. perfringens, C. septicum, or a C. difficile NAP1 strain.

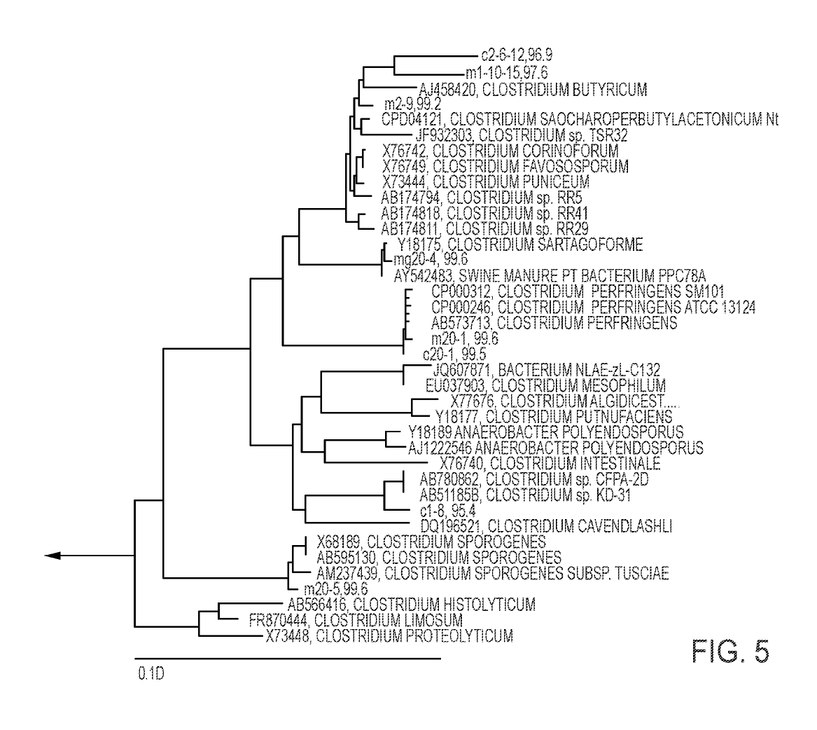

To identify these isolates taxonomically, we sequenced their 16S rRNA genes and used several of the most up to date and comprehensive databases to identify the most closely related known sequences. Eight unique isolates belonged to the Clostridiales family (FIG. 5) and were selected for further characterization. In particular, two axenic cultures represent potentially novel species within Cluster I of the genus Clostridium using the accepted species definition of <97% sequence identity to the closest relative. Strain c2-6 is most closely related to C. butyricum (96.9% identity) and strain c1-8 most closely related to Clostridium sp. KD-31 (95.4% identity; FIG. 5). For each new isolate cultivated here (shown in bold or red font), the strain name is shown followed by the percent identity of the 16S gene to its nearest relative. The two strains in red (FIG. 5) represent potentially novel species based on accepted rules of species cutoffs at <97% identity.

Finally, to further characterize these two potentially novel Clostridia species, their entire genomes were sequenced. Genome sequencing of these two strains has so far revealed several interesting characteristics including several bacteriophage holin and endolysin genes. From the genome sequences, it was possible to obtain the sequences for another taxonomic marker gene (cpn60, a housekeeping gene). This analysis showed that c2-6 is most closely related to C. accharoperbutylacetonicum and C. beijerinckii, and c1-8 is most similar to the chitinolytic C. araputrificum, C. celatum, and C. butyricum.

### Example 3

Cultivation-Based Selective Enrichment Scheme of Potential Probiotic Bacteria.

A selective cultivation scheme was undertaken to selectively isolate Clostridia from healthy chicken ceca. approximately 40 strains were brought into axenic culture as described in the previous Example. Briefly, exposure to formaldehyde, ethanol, and/or heat were used to kill vegetative cells and select for spore-forming bacteria plated on a variety of growth media. Preliminary phylogenetic placement (based on 16S rRNA gene sequences) of isolates obtained showed that most strains belong to the Clostridiales. Of particular interest are some potentially novel taxa identified based on low (<97% sequence similarity cutoff as rule-of-thumb for species distinctions) 16S rRNA homology to the nearest known relatives.

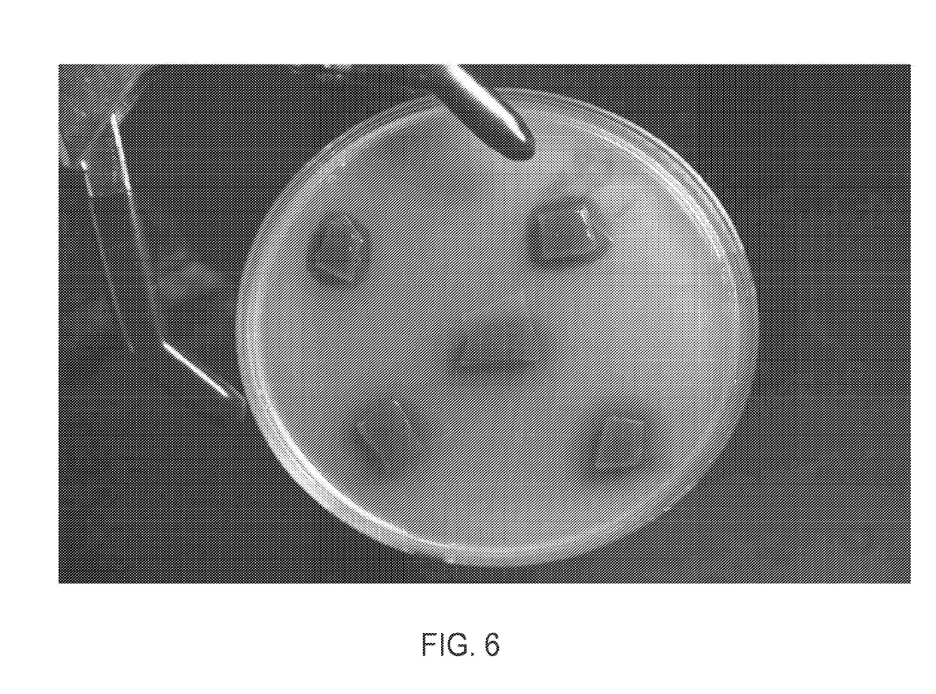

To assess their potential for competitive exclusion of pathogens, newly-recovered isolates were tested against a panel of common gut pathogens. Growth inhibition of at least one of the known pathogens C. perfringens, C. septicum, or a C. difficile NAP1 strain was observed for eight of our novel isolates from ceca and five from mid-gut samples (FIG. 6). Putative novel isolates c2-6 and c1-8 (upper left and upper right of FIG. 8) demonstrated anti-microbial properties when placed on a lawn of C. perfringens as demonstrated by zones of clearance. As reported in the previous Example, it was possible to get the sequences for another taxonomic marker gene (cpn60, a housekeeping gene), which showed that c2-6 is most closely related to C. saccharoperbutylacetonicum and C. beijerinckii, and c1-8 is most similar to the chitinolytic C. paraputrificum, C. celatum, and C. butyricum (FIG. 5).

### Example 4

Probiotic Application to the Poultry Litter.

Chlorine dioxide (ClO2) granules were applied to the litter to reduce microbial population. Approximately 50 chicks per treatment (200 total) were placed in a two-way factorial design with litter treated and untreated with ClO2 and probiotic addition. Probiotic cultures were obtained by serial passaging cecal contents through several sets of chicks, harvesting or obtaining the cecal contents at one week of age and using it as an oral gavage for the next set of chicks.

What is claimed is:

1. A composition suitable for oral administration to a young poultry bird comprising: (a) cecal material from adult poultry birds and (b) saline, wherein the ratio of (a) to (b) ranges from 1:2 to 1:4 (v/v).

2. The composition of claim 1, wherein the cecal material is from adult chickens, wherein the ratio of (a) to (b) is 1:3, and wherein the composition is suitable for oral administration to a young chicken.

3. The composition of claim 1, wherein the composition further comprises one or more of the following: a bacterial community significantly enriched in Bacteroides, a nutrient, an amino acid, a vitamin, a mineral, and a fatty acid.

4. A method of increasing body weight gain in a poultry bird comprising administering about 0.2 mL of the composition of claim 1 to a young poultry bird by oral gavage.

5. A method of increasing body weight gain in a chicken comprising administering about 0.2 mL of the composition of claim 2 to a young chicken by oral gavage.

6. The method of claim 5, wherein the young chicken is a day-of-hatch chick.

7. A method of increasing feeding efficiency in a poultry bird comprising administering about 0.2 mL of the composition of claim 1 to a young poultry bird by oral gavage.

8. A method of increasing feeding efficiency in a chicken comprising administering about 0.2 mL of the composition of claim 2 to a young chicken by oral gavage.

9. The method of claim of claim 8, wherein the young chicken is a day-of-hatch chick.

10. A composition suitable for application to poultry litter, wherein the composition is prepared by (a) obtaining the cecal material from adult poultry birds; (b) administering the cecal material to young poultry birds by oral gavage; (c) obtaining the cecal material from the inoculated poultry birds and subjecting it to serial passaging through multiple generations of birds; (d) obtaining the serially-passaged cecal material; and (e) suspending the serially-passaged cecal material in saline.

11. A composition suitable for application to chicken litter, wherein the composition is prepared by (a) obtaining the cecal material from adult chickens; (b) administering the cecal material to young chickens by oral gavage; (c) obtaining the cecal material from the inoculated chickens and subjecting it to serial passaging through multiple generations of birds; (d) obtaining the serially-passaged cecal material; and (e) suspending the serially-passaged cecal material in saline.

12. The composition of claim 11, wherein the young chickens in step (b) are day-of-hatch chicks and wherein the serial passaging in step (c) is done through at least 3 sets of chicks.

13. A method of preventing or reducing colonization of the chicken gut by pathogenic bacteria comprising the step of applying the composition of claim 11 to the chicken litter.

14. A method of preventing or reducing colonization of poultry gut by pathogenic bacteria comprising the step of applying the composition of claim 1 to the poultry litter.

15. The method of claim 14, wherein the composition is applied to the poultry litter by aerosol spraying, and wherein the aerosol spray comprises about 10⁸ probiotic cells per cm².

16. A litter composition comprising poultry litter and a composition comprising: (a) cecal material from adult poultry birds and (b) saline, wherein the ratio of (a) to (b) ranges from 1:2 to 1:4 (v/v).

17. A method of selecting for bacterial strains with probiotic potential comprising the steps of: a) mixing a suspension of cecal contents with chloroform to select for spore-forming bacteria and b) removing the chloroform from the mixture by centrifugation and washing with saline.

18. A composition suitable for application to the chicken litter, wherein the composition is prepared by (a) obtaining the cecal material from adult chickens; (b) administering the cecal material to young chickens by oral gavage; (c) obtaining the cecal material from the inoculated chickens and subjecting it to serial passaging through multiple generations of birds; (d) obtaining the serially-passaged cecal material; (e) suspending the serially-passaged cecal material in saline; (f) mixing the suspension of the cecal material with chloroform to select for spore-forming bacteria; (g) removing the chloroform from the mixture by centrifugation; and (h) washing with saline, wherein the composition comprises one or more of the following strains of Clostridia: c2-6-12; c1-8; m1-10-15; m2-9; mg20-4; m20-1; c20-1; and m20-5.
